(19) United States
(12) Patent Application Publication
(10) Pub. No.: US 2004/0055029 A1
(43) Pub. Date: Mar. 18, 2004

(54) Genetically distinct strain of channel catfish designated NWAC103, with improved growth performance

(76) Inventors: William R. Wolters, Cleveland, MS (US); Geoffrey C. Waldbieser, Cleveland, MS (US); Brian G. Bosworth, Cleveland, MS (US); Jeffrey T. Silverstein, Middletown, MD (US); Edwin H. Robinson, Starkville, MS (US)

Correspondence Address: Supervisor, Patent Prosecution Services, PIPER RUDNICK LLP, 1200 Nineteenth Street, N.W., Washington, DC, 20036-2412, US

(21) Appl. No.: 10/245,698

(22) Filed: Sep. 18, 2002

(51) Int. Cl. A01K 67/027

(52) U.S. Cl. 800/20; 119/200

(57) ABSTRACT

A substantially purebred non-transgenically developed fish produced by selecting fish having at least one desirable trait from a population of same species fish and preparing a DNA fingerprint of the selected fish so as to be able to identify breeder fish by use of selected microsatellite loci identified as being associated with fish having the at least one desired trait, breeding the selected breeder fish to produce offspring having the at least one desired trait. Also provided is a method for producing the substantially purebred non-transgenically developed fish.

This invention was made with Government support under 99-34311-7539 awarded by the U.S. Department of Agriculture. The Government may have certain rights in this invention.

### BACKGROUND OF THE INVENTION

1. Field of the Invention

The invention relates to a substantially purebred non-transgenically developed, distinct strain of channel catfish (Ictalurus punctatus) with increased growth rate.

2. Background of the Invention Technology

Efforts have been made in the past to use transgenic technology to develop a salmonid fish with a higher growth rate. This fish is a cold-water fish, which is not adapted to the environmental conditions found in commercial warm-water aquaculture operations in the lower southeastern United States. In contrast, the present invention employs non-transgenic methods to develop an ictalurid fish (a warm-water fish) which is adapted to environmental conditions found in commercial warm-water aquaculture operations in the lower southeastern United States.

By using non-transgenic methods the invention does not have the regulatory restrictions associated with transgenic (i.e., genetically-modified) organisms. As such, it does not have to overcome the negative perceptions often associated with genetically-modified organisms, which should improve its acceptance in the marketplace.

There has been limited genetic improvement of channel catfish stocks in commercial culture. Most farmers are utilizing catfish stocks that are relatively unselected for commercially important traits compared to wild stocks. This is true for most warm-water aquatic species that are commercially cultured in the United States. Thus the present invention represents a major improvement in this commercially important species.

### SUMMARY OF THE INVENTION

The present invention provides a substantially purebred non-transgenically developed fish having at least one identifiable trait and useful as breeding stock, the fish being produced by a process that includes selecting potential breeder fish that demonstrate the identifiable trait from a population of same species fish, preparing at least a partial DNA fingerprint of the selected potential breeding stock, identifying those fish with the potential breeding stock that have specific DNA micorsatellite loci which distinguish and identify the selected breeding stock from all other fish, breeding the selected breeding stock so as to produce the substantially purebred non-transgenically developed breeding stock fish.

The present invention also provides a substantially purebred non-transgenicially developed fish having desirable traits and useful as breeding stock, which is produced by a process that includes selecting at least one fish from a group of same species fish, the selected fish having the desirable traits. Creating at least a partial DNA fingerprint for the at least one selected fish whereby variations at a number of microsatellite loci can be identified so as to be able to easily identify other fish having the same desirable traits. Selecting breeding stock having the desirable traits by using the DNA fingerprinting system and selectively breeding the breeding stock so as to produce the substantially purebred breeding stock fish having the desired traits.

The present invention also provides a method of developing substantially purebred breeding stock from a population of same species fish that includes, selecting potential breeding stock that have the phenotype of at least one desired trait from the population; genetically analyzing tissue sample of the potential breeding stock and comparing the same to a DNA fingerprint of the fish species so as to identify selected microsatellite loci found only in fish having the desired trait, such fish are then bred so as to produce substantially purebred breeding stock fish.

### DETAILED DESCRIPTION OF THE INVENTION

Conventionally selection of breeding stock in commercial fish farming is accomplished by sight-selection and thus a wide variance in breeding stocks is normal.

Catfish stocks were originally obtained from the U.S. Fish and Wildlife Service National Fish Hatchery in Uvalde, Tex. Sub-adult fish (1992 year class—F0 generation) were obtained in 1993 and reproduced in 1994 as 2-year old broodfish. Subsequent generations were produced and selected from the offspring of 2-year old spawners. Full-sibling families (F1 generation) obtained in 1994 were evaluated for enteric septicemia (ESC) resistance and family selection was performed in 1994 for that characteristic, and within family selection was performed for growth rate, and those fish saved as future broodfish. These offspring (F2 generation—1998 and 1999 year classes) were then cultured in earthen ponds at the Thad Cochran National Warmwater Aquaculture Center in Stoneville, Miss.

A DNA fingerprinting system was developed to identify the NWAC103 channel catfish based on DNA sequence variation at microsatellite loci. Genomic DNA isolated from a blood sample or a small tissue sample was amplified using the polymerase chain reaction, and DNA fragment size determined by electrophoresis. Variation in microsatellite alleles was characterized in 3 generations of catfish from the NWAC103 fry; fish from 20 fingerling operations in Mississippi, Alabama, Arkansas, and Louisiana; and wild fish from the Mississippi River. Based on inheritance and genotype frequencies from ten microsatellite markers, the system of this invention can distinguish NWAC103 catfish from non-NWAC103 catfish. Based on a random sample of 96 fish, the probability of finding two “off-type” fish with NWAC103 genotypes is 1 in 59 million. The probability of finding two NWAC103 fish with “off type” genotypes (due to estimated spontaneous mutation) is 1 in 100 million fish. Thus, the present invention provides a substantially purebred breeding stock fish as compared to the conventional wide variance in fish farming breeding stock. The substantially purebred breeding stock produced by the present invention are genetically more purebred than the conventionally produced and selected breeding stock in the catfish farming industry. Based on the microsatellite loci identification method of the invention, purebred breeding stock can be produced that are at least 90% purebred.

The physical characteristics and rearing conditions of catfish lead to problems in identification of individual fish or strains. Physical tagging methods are impractical for large numbers of fish, so an endemic marking system is required. Variation in DNA sequence between individuals can be used as a fingerprinting tool for identification, and inheritance of DNA sequences is useful for determining family structure. A class of DNA markers termed “microsatellites” consists of short repeated sequence motifs within the long stretches of genomic DNA. The region of DNA flanking and including a microsatellite location (locus) can be copied in a rapid laboratory test that uses polymerase chain reaction technology (PCR), creating millions of copies of the defined region. Changes in the number of repeats at the microsatellite locus result in differences in the length of the PCR product, and one or two differently sized products (alleles) are possible from one fish because it has inherited a copy of every locus from each parent. The PCR reactions and measurement of the PCR fragment length are both standard molecular biology procedures, and PCR technology allows collection of very small tissue samples.

Microsatellite loci were cloned from channel catfish and 313 loci have been characterized in channel catfish populations (See Waldbieser et al., 2001, the complete disclosure of which is fully incorporated herein by reference, See Table 1). The DNA primers used to analyze these loci are now commercially available from ResGen (http://www.resgen.com/products/ADDMP pf.php3; Invitrogen, Carlsbad, Calif.). Eleven loci were selected for use as genetic markers for strain verification. Genomic DNA was prepared from full siblings of the released NWAC103 strain catfish when the siblings were fry. Genomic DNA was also prepared from 24 fry from each of 20 commercial fingerling operations. Allele sizes were determined using an ALF Express DNA Analysis System (Pharmacia Biotech, Piscataway, N.J.). Alleles were scored for each fish for each marker (Table 2.), and all possible combinations of two alleles (genotypes) were calculated to determine genotype frequencies. When a NWAC103 allele was not found in commercial populations, a conservative value of 5% was used as the allele frequency to account for sampling error. The proportion of commercial fish exhibiting NWAC103 genotypes is presented in Table 3.

When a catfish is randomly sampled and genotyped, one of four decisions are made based on inclusion or exclusion as a NWAC103 catfish (Table 4.). If the catfish is a NWAC103 and displays a NWAC103 genotype, then it is correctly included. If the catfish it not a NWAC103 and displays alleles not found in NWAC103 catfish, then it is correctly excluded. A non-NWAC103 catfish could be falsely included as a NWAC103 if the microsatellite alleles at all 11 loci are the same as those found in the NWAC103 population. Using the genotype frequencies calculated from commercial catfish, the probability of a randomly selected commercial catfish having a genotype identical to a NWAC103 is 1 in 85,470 (false inclusion). A NWAC103 fish would be excluded from certification if two markers underwent spontaneous de novo DNA repeat expansion or deletion. Microsatellite-repeat mutation rates range from 1 in 100 to 1 in 1 million. Microsatellite loci with a repeat motif of two bases (e.g. CA, AT) mutate more frequently than those with three (e.g. AAT, AAC) and four basepair (e.g. GATA, AAAT) repeat motifs. The catfish markers contain three and four basepair repeat motifs. Using a conservative mutation rate estimate of 1 in 500, the probability of a NWAC103 fish undergoing two de novo mutations is 1 in 250,000 (false exclusion).

TABLE 1

Polymorphic Channel Catfish Microsatellite
Loci From Genomic libraries.

| | | | |
|---|---|---|---|
| | Alleles^{a} | | |
| Locus | Min/Max | No. | Primers |

| | | | | |
|---|---|---|---|---|
| IpCG0002 | 214/247 | 10 | 5′-CCACAAGGTTTAGGGCATCA-3′ | (SEQ ID NO.1) |
| | | | 5′-TGAGTACAGCGCTTTGAG-3′ | (SEQ ID NO.2) |
| IpCG0032 | 273/321 | 16 | 5′-GTTACAATATTTAGGAACGGTATAAGC-3′ | (SEQ ID NO.3) |
| | | | 5′-TAAGATGCGTATGAAGACAAACCC-3′ | (SEQ ID NO.4) |
| IpCG0035 | 291/345 | 20 | 5′-AACCACTAAGCCTAGCACGTTC-3′ | (SEQ ID NO.5) |
| | | | 5′-AGTATGGGTACTGCAACAAAACAAG-3′ | (SEQ ID NO.6) |
| IpCG0038 | 105/161 | 13 | 5′-GTGTGCCTGATTTACTAATGATAAG-3′ | (SEQ ID NO.7) |
| | | | 5′-TGTATTGGTATAGAACACATTAGCC-3′ | (SEQ ID NO.8) |
| IpCG0070 | 218/310 | 28 | 5′-ATCATTTTCTGCTTCTTATACATAGGCT-3′ | (SEQ ID NO.9) |
| | | | 5′-CCTTTAGATGAACTCACCTGCC-3′ | (SEQ ID NO.10) |
| IpCG0128 | 256/324 | 17 | 5′-GATCCACTGAGAAATAAGAGCACA-3′ | (SEQ ID NO.11) |
| | | | 5′-GGAGTATAGCACAGAAACACGAA-3′ | (SEQ ID NO.12) |
| IpCG0189 | 219/261 | 14 | 5′-GATCCTGTGCTAAAGAAACCAAG-3′ | (SEQ ID NO.13) |
| | | | 5′-GTGCCGCAGTGTGTTGTAAA-3′ | (SEQ ID NO.14) |
| IpCG0195 | 220/249 | 15 | 5′-GCAGGTCTGTCGTCATCTAC-3′ | (SEQ ID NO.15) |
| | | | 5′-AACTGTCATTTACACACATTCATCTA-3′ | (SEQ ID NO.16) |
| IpCG0211 | 155/188 | 11 | 5′-GCCTCCCGAGCCTCCAAAACA-3′ | (SEQ ID NO.17) |
| | | | 5′-CTGTGATGGTGCCCTTTTCTTAC-3′ | (SEQ ID NO.18) |
| IpCG0256 | 125/176 | 18 | 5′-TTTGTTCAACAGCTTGCTCG-3′ | (SEQ ID NO.19) |
| | | | 5′-CCAATGTTAAATGATGTTCATCG-3′ | (SEQ ID NO.20) |
| IpCG0273 | 143/191 | 15 | 5′-CGTTTTACTTCCTCATACAGCAC-3′ | (SEQ ID NO.21) |
| | | | 5′-GCACCAAGAGACCTGTGACA-3′ | (SEQ ID NO.22) |

^{a}Smallest and largest alleles (in base pairs), and number of alleles in catfish populations

TABLE 2
Microsatellite alleles (in base pairs) found in
commercial and NWAC103 catfish

IpCG0002
214*
217*
220*
223*
226
232
238
241
244
247
IpCG0032
273
275*
277*
283
285
289
293*
295*
297*
299
301*
305
309
313*
317*
321
IpCG0035
291
295
299*
303*
307*
311*
313
315
317
319
321
323
325
327
329
333
337
339
341
345
IpCG0038
105*
109
113*
117*
121
133*
137
141
145
145
149
153
161
IpCG0070
218
226
228
230*
232
234
236
238*
242*
244
246
250*
254
258*
262
266*
270
272*
274
278
282
286*
290*
294
296
298
302
310
IpCG0128
256*
260*
264
268*
272
276*
284
288*
292
296
300*
302
304*
312
316
320*
324*
IpCG0189
219
225
228
231*
234*
237*
240*
243*
246*
249
252
255*
258
261
IpCG0195
220
226
229*
231
232
234
235
236
237
238
240
241
243*
246*
249*
IpCG0211
155
158
161*
164*
167*
170*
173
176
179
182
188
IpCG0256
125
128*
131
134*
137*
140*
143*
146*
149
152
155
158
161
164
167
170
173
176
IpCG0273
143*
149*
152*
155
158
161
164*
167
170
173*
176
182
185
188
191
*Alleles specific to NWAC103 catfish.

TABLE 3
Frequency of NWAC103 genotypes in commercial catfish.

| | Proportion of farm |
|---|---|
| Locus | fish with NWAC103 genotype |
| IpCG0002 | 0.59 |
| IpCG0032 | 0.47 |
| IpCG0035 | 0.26 |
| IpCG0038 | 0.44 |
| IpCG0070 | 0.24 |
| IpCG0128 | 0.39 |
| IpCG0189 | 0.43 |
| IpCG0195 | 0.08 |
| IpCG0211 | 0.57 |
| IpCG0256 | 0.56 |
| IpCG0273 | 0.36 |
| Combined | 0.0000117 |

TABLE 4
Probability of error in classifying one randomly sampled
catfish as belonging to NWAC103 strain.

Sample

| Genotype | NWAC103 | Other |
|---|---|---|
| NWAC103 | Correct inclusion | Incorrect inclusion |
| | | 1/85,000 |
| Other | Incorrect exclusion | Correct exclusion |
| | 1/250,000 | |

Performance

Reproductive Performance.

Some NWAC103 females spawned at an early age (2 years), and overall demonstrated high spawning success and fecundity (smaller egg size). Elevated reproductive steroid levels found in NWAC103 fish may be indicative of early sexual maturity and the probability for spawning success (See Examples section Tables 8-10).

Tank Studies.

Seven different studies were conducted to compare growth performance, carcass composition, and serum hormone levels of NWAC103 catfish versus other catfish. In one or more of the studies, five other catfish stocks, five dietary protein levels, and effect of two culture temperatures were evaluated (See Examples section, Tables 11-19). These studies were carefully controlled with large numbers of replicated tanks and primarily utilized juvenile fish, however, one tank study cultured juvenile fish to marketable size. In all seven tank studies, the NWAC103 catfish demonstrated significantly faster growth and better feed conversion than other catfish (Table 5). In six of seven tank studies, NWAC103 catfish consumed significantly more feed. No significant differences were found for survival and carcass composition. Serum levels of insulin-like growth factor-1 (IGF-1), a hormone regulating growth, were significantly higher and correlated with faster growth at both 21.7° C. and 26.0° C. in NWAC103 catfish compared to one commercial catfish line. Serum estrogen levels in sub-adult NWAC103 female fish were higher than females from one commercial catfish line and may be an indication of earlier sexual maturity in NWAC103 catfish. NWAC103 catfish were found to be susceptible to enteric septicemia infection as are all channel catfish. NWAC103 catfish were less susceptible than Norris catfish in one study and more susceptible in another study (See Example section Tables 20 and 21).

Table 5 shows overall results from seven tank studies comparing NWAC103 catfish (least squares mean+pooled SEM and probability of difference) versus all other catfish.

TABLE 5

| | | Other | | |
|---|---|---|---|---|
| Variable | NWAC103 | catfish | Difference | Probability |
| Specific growth rate | 2.7 ± 0.2 | 2.1 ± 0.2 | +22% | 0.02 |
| (% increase | | | | |
| in weight/day) | | | | |
| Food consumption | 13.1 ± 0.4 | 11.9 ± 0.4 | +9% | 0.02 |
| (% food/day | | | | |
| based on initial wt) | | | | |
| Food conversion | 1.30 ± .10 | 1.64 ± .10 | +20% | 0.01 |
| Overall survival | 99.1 ± 0.4 | 99.2 ± 0.4 | — | 0.85 |
| Protein | 17.1 ± 0.5 | 17.6 ± 0.5 | — | 0.50 |
| Fat | 5.4 ± 0.6 | 4.2 ± 0.6 | — | 0.26 |
| Moisture | 76.3 ± 0.5 | 77.3 ± 0.5 | — | 0.11 |

Pond Studies:

Growth performance, carcass composition, and fillet yield of NWAC103 catfish versus eight other catfish lines were compared at three research locations. Three studies were conducted with NWAC103 catfish cultured communally with other lines stocked into the same replicated ponds. Eight pond studies cultured each catfish line in separate ponds. All pond studies comparing NWAC103 catfish to other catfish were for one growing season in batch culture. In one or more of the studies, six other catfish lines and three dietary protein levels were evaluated at three research locations.

Communal Stocking Studies:

All three studies cultured fingerlings to marketable size. Harvest weight was significantly larger in NWAC103 catfish than other catfish in all three communal studies (Table 6 and Examples section Tables 22-24). Specific growth rate (% increase in weight/day) was greater than all other catfish in two out of three studies. The large difference in harvest weight between NWAC103 catfish and other catfish apparent in these studies is likely the result of a competitive advantage from vigorous feeding activity in NWAC103 catfish and higher food consumption (also found in tank studies) and in effect restricting the feed available to less aggressive catfish. No differences were found for survival or fillet yield in communal studies.

Table 6 shows the overall results from pond communal stocking studies comparing NWAC103 catfish (least squares mean+pooled SEM and probability of difference) versus all other catfish.

TABLE 6

| Variable | NWAC103 | Other catfish | Difference | Probability |
|---|---|---|---|---|
| Harvest | 1.01 ± 0.2 | 0.50 ± 0.2 | +50% | 0.01 |
| Weight (lbs) | | | | |
| Specific | 1.12 ± 0.1 | 1.02 ± 0.1 | +9% | 0.07 |
| growth rate | | | | |
| (% increase | | | | |
| in weight/day) | | | | |
| Overall survival | 87.3 ± 6.4 | 85.4 ± 6.4 | — | 0.78 |
| Fillet yield (%) | 44.8 | 43.5 | — | 0.98 |

Separate Stocking Studies:

One study cultured fry to fingerlings and the other seven studies cultured fingerlings to marketable size (See Examples Section Tables 25-33). Higher harvest weight and feed consumption were found for NWAC103 catfish fry cultured to fingerlings, but no differences were found in yield, feed conversion or survival (Table 7). In all other pond studies, NWAC103 catfish had significantly higher harvest weight and gain. Yield was higher in five studies, not different in one study, and lower in another as a result of overwinter mortality. Survival was not different in three studies, but lower in three studies. Cause of mortality was usually not a result of known disease. Lower survival may have resulted from low pond chloride levels in two studies, because mortalities ceased after sodium chloride was added to experimental ponds that raised levels to 100 ppm. Feed conversion was not different in four studies, but was lower in the three studies where survival was lower. No differences were found for fillet yield compared to other channel catfish. Fillet yield for blue catfish x channel catfish hybrids was significantly greater (˜2%) than for channel catfish.

Table 7 shows the overall results from separate stocking studies comparing NWAC103 catfish (least squares mean+pooled SEM and probability of difference) versus all other catfish cultured from fingerling to marketable size.

TABLE 7

| Variable | NWAC103 | Other catfish | Difference | Probability |
|---|---|---|---|---|
| Harvest | 1.35 ± 0.07 | 1.04 ± 0.07 | +23% | 0.01 |
| Weight (lbs) | | | | |
| Specific | 1.50 ± 0.04 | 1.39 ± 0.04 | +7% | 0.02 |
| growth rate | | | | |
| (% increase | | | | |
| in weight/day) | | | | |
| Feed | 10,968 ± 621 | 9,505 ± 621 | +13% | 0.05 |
| consumption | | | | |
| (lbs/acre) | | | | |
| Yield (lbs/acre) | 5,927 ± 591 | 5,341 ± 621 | — | 0.33 |
| Feed | 1.74 ± 0.1 | 1.61 ± 0.1 | — | 0.33 |
| conversion | | | | |
| efficiency | | | | |
| Overall | 82.8 ± 6.4 | 87.5 ± 6.4 | — | 0.49 |
| survival | | | | |
| Fillet yield (%) | 44.9 ± 1.9 | 44.9 ± 1.9 | — | 0.99 |

Results of experimental trials have shown the NWAC103 catfish has excellent growth and reproductive traits compared to other catfish currently being used by producers. The growth advantage of NWAC103 catfish appears to be due to aggressive feeding behavior and higher feed consumption. NWAC103 fish should reach market weight faster than fish currently cultured. Optimum growth and high production of NWAC103 catfish necessitates following recommended management guidelines and maintaining optimum environmental conditions. Recommendations for optimizing performance of NWAC103 line catfish are given following the Examples Section. Realized performance in commercial production may vary from experimental results due to differences in management strategies. Joint release and commercial utilization of NWAC103 catfish should benefit commercial catfish farmers, catfish processors, and consumers.

### EXAMPLES

Summaries of research studies followed by tables of mean performance data for NWAC103 catfish versus other catfish from replicated experimental studies in tanks, ponds, and culture locations or environments. Means followed by the same letter are not significantly different.

NWAC103 catfish broodfish were significantly larger than the Kansas catfish in this study, but stocking densities were adjusted to equalize the stocking rate (lbs/acre). The spawning success was significantly higher in the NWAC103 catfish, but no differences were found for hatching percentage. Egg size was significantly smaller and probably results in higher fecundity. Fecundity of individual spawning females was calculated after determining parentage of individual spawns from molecular markers. Fecundity was not significantly greater, however, NWAC103 broodfish probably have larger fecundity at a common weight because catfish fecundity generally decreases as fish size increases. Testosterone concentrations in male NWAC103 fish were 18.6+0.08 compared to 0.58+0.18 ng/ml (mean+SE) for the Kansas catfish. Estrogen and testosterone concentrations in female NWAC103 fish were 9.68+0.83 and 9.42+1.88 compared to 0.95+0.08 and 0.36+0.11 ng/ml for female Kansas fish. Spawning success was higher in NWAC103 catfish. The difference in spawning success may be explained by differences in sex hormone concentrations. There were no significant differences of sex hormone concentrations between fish which spawned and those which did not spawn, however, steroid levels may be indicative of probability for spawning success and sexual maturation.

Table 4 shows the mean reproductive characteristics of four channel catfish lines from data collected during the 1996 spawning season at the USDA/ARS Catfish Genetics Research Unit, Stoneville, Miss.

TABLE 8

| | USDA102 | NWAC103 | Kansas | Norris | Blue Catfish^{b} |
|---|---|---|---|---|---|
| Age (years) | 4 | 4 | 4 | 4 | NA |
| Spawn Weight (kg) | 0.58 ± 0.08 | 0.65 ± 0.08 | 0.36 ± 0.05 | 0.63 ± 0.05 | 1.55 ± 0.00 |
| Egg size | 29.45 ± 1.28 | 34.56 ± 1.13 | 29.08 ± 1.08 | 28.05 ± 0.98 | 34.00 ± 0.00 |
| (#eggs/gram) | | | | | |
| Number | 16,359 ± 2,228 | 21,889 ± 2,434 | 11,054 ± 1,468 | 17.422 ± 1.293 | 52,700 ± 0.00 |
| eggs/spawn | | | | | |
| Fry Weight | 0.125 ± 0.003 | 0.122 ± 0.002 | 0.123 ± 0.003 | 0.135 ± 0.005 | 0.140 ± 0.000 |
| at 24 hr | | | | | |
| Fry Weight | 0.138 ± 0.004 | 0.142 ± 0.005 | 0.142 ± 0.006 | 0.141 ± 0.005 | 0.120 ± 0.000 |
| at 7 days | | | | | |
| Fry Weight | 0.228 ± 0.017 | 0.284 ± 0.048 | 0.272 ± 0.027 | 0.205 ± 0.010 | 0.187 ± 0.000 |
| at 14 days | | | | | |
| Female Weight | NA | 3.49 ± 0.08 | 1.38 ± 0.01 | 1.86 ± 0.10 | NA |
| (kg) | | | | | |
| Fecundity | NA | 4,112 ± 376 | 3,355 ± 413 | 4,872 ± 218 | NA |
| (#eggs/lb female) | | | | | |

^{b}Only 1 blue catfish spawn obtained in 1996;
NA = data not available.

Table 9 shows the mean (+SE) reproductive characteristics of NWAC103 and Kansas broodfish spawned in replicate 0.1-acre earthen ponds during the 1996 spawning season at the USDA/ARS Catfish Genetics Research Unit, Stoneville, Miss.

TABLE 9

| Variable | Kansas | NWAC103 |
|---|---|---|
| Female weight (kg) | 1.38 ± 0.02a | 3.62 ± 0.13b |
| Male weight (kg) | 1.56 ± 0.05a | 3.54 ± 0.20b |
| Stocking rate (lbs/acre) | 1,796 ± 28a | 1,652 ± 38a |
| Sex ratio (♀:♂) | 1.5:1.0 | 1.25:1.0 |
| Spawning success | | |
| Female (%) | 23.3 ± 5.1a | 57.5 ± 7.6b |
| Male (%) | 20.0 ± 2.9b | 50.0 ± 0.0b |
| Hatch (%) | 51.6 ± 9.4a | 42.4 ± 7.6a |
| Egg size (# eggs/gram) | 29.1 ± 1.1a | 33.8 ± 1.3b |
| Absolute fecundity (# eggs/lb) | 3,355 ± 413a | 3,939 ± 394a |
| Testosterone males (ng/ml) | 0.58 ± 0.18a | 18.6 ± 1.54b |
| Estrogen females (ng/ml) | 0.95 ± 0.08a | 9.68 ± 0.83b |
| Testosterone females (ng/ml) | 0.36 ± 0.11a | 9.42 ± 1.88b |

Table 10 provides a summary of reproductive characteristics of breeder class NWAC103 catfish cultured in earthen ponds at the USDA/ARS Catfish Genetics Research Unit, Stoneville, Miss.

TABLE 10

| Variable | 1994 - Age 2 | 1995 - Age 3 | 1996 - Age 4 |
|---|---|---|---|
| Pond | 0.25 acre (n = 1) | 0.1 acre (n = 3) | 0.1 acre (n = 3) |
| Number | 396/acre | 100/acre | 60/acre |
| of Males | | | |
| Number | 240/acre | 170/acre | 100/acre |
| of Females | | | |
| Mean | 0.80 lb | 5.46 ± 0.09 | 8.40 ± 0.37 |
| Weight Males | | | |
| Mean | 0.70 lb | 4.37 ± 0.20 | 7.86 ± 0.17 |
| Weight Females | | | |
| Stocking | 485 | 1,289 ± 43 | 1,124 ± 21 |
| Rate (lbs/acre) | | | |
| Sex Ratio | 1.65♀:1.0♂ | 1.7♀:1.0♂ | 2.23♀:1.0♂ |
| Spawning % | 28.0 | 54.9 ± 3.9 | 73.3 ± 8.8 |
| Number | 13,751 | 10,746 ± 1,136 | 3,757 ± 400 |
| Eggs/lb of | | | |
| Female | | | |
| Spawned | | | |
| Number of | 3,889 | 5,811 ± 158 | 2,809 ± 106 |
| Eggs/lb of | | | |
| Female | (661,164/acre) | (2,353,210/acre) | |
| Stocked | | | |
| Hatching % | 28.0 | 52.0 ± 9.16 | 46.4 ± 7.1 |
| Number | 8,154 | 5,422 ± 601 | 1,743 ± 186 |
| of Fry/lb of | | | |
| Female | (392,048/acre) | (1,213,830/acre) | (836,191/acre) |
| Spawned | | | |

As shown in Table 11, regardless of dietary protein concentration, there were differences in weight gain and feed consumption among lines with highest weight gain and feed consumption being observed in NWAC103 channel catfish, and lowest weight gain and feed consumption observed in those of Mississippi-normal (MN) catfish. NWAC103 catfish converted the feeds better than those of other two catfish lines. Regardless of channel catfish line, differences in weight gain, feed consumption, and FCR were observed among fish fed diets containing various levels of protein with the 35% protein diet being the best. Mean survival of fish was 98% to 100% which were not different among treatments. Using pooled data by fish line, highest fillet protein was found in USDA102 catfish, which was higher than that of USAD103 catfish, but not more than that of the MN catfish. Lowest fillet fat was found in MN catfish, which was probably due to the smaller size and slower growth rate. Using pooled data by dietary protein concentration, there were no differences in fillet protein among fish fed different concentrations of dietary protein. Fish fed the 45% protein diet had lowest fillet fat, which was lower than fish fed the 35% protein diet, but not different from fish fed the 25% protein diet. NWAC103 catfish demonstrated the fastest growth rate, highest food consumption and best feed efficiency of the three catfish lines tested in the study. Growth rate is one trait that has been selected in this line, and performance did exceed USDA102 catfish (another line being selected and evaluated for release) and MN catfish which is currently used in commercial culture.

Table 11 shows mean weight gain, feed consumption, feed conversion ratio (FCR), survival and proximate composition of three channel catfish lines fed diets containing three levels of protein in replicated experimental tanks.

TABLE 11

| | Protein | Weight gain | Consumption | | Survival | Protein | Fat | Moisture | Ash |
|---|---|---|---|---|---|---|---|---|---|
| | (%) | (g/fish) | (g/fish) | FCR | (%) | (%) | (%) | (%) | (%) |
| Line | | | | | | | | | |
| NWAC103 | 25 | 59.8 b | 100.4 bc | 1.69 c | 99.0 a | 17.1 a | 3.9 a | 77.8 bcd | 1.09 abc |
| | 35 | 87.8 a | 115.9 a | 1.32 d | 100.0 a | 17.1 ab | 4.0 a | 77.7 cd | 1.06 c |
| | 45 | 67.2 b | 114.0 a | 1.70 c | 100.0 a | 16.8 ab | 3.3 abc | 78.6 abc | 1.01 d |
| USDA102 | 25 | 42.4 c | 94.9 d | 2.25 b | 100.0 a | 17.3 ab | 3.3 abc | 78.3 bcd | 1.12 ab |
| | 35 | 64.3 b | 104.9 b | 1.65 c | 100.0 a | 17.9 a | 3.8 ab | 77.4 d | 1.11 abc |
| | 45 | 48.0 c | 100.9 bc | 2.11 b | 100.0 a | 17.7 a | 3.0 bcd | 78.1 bcd | 1.09 bc |
| MN | 25 | 32.2 d | 83.0 e | 2.60 a | 98.0 a | 16.4 b | 2.4 d | 79.7 a | 1.14 a |
| | 35 | 59.6 b | 101.5 bc | 1.71 c | 100.0 a | 17.5 ab | 2.8 cd | 78.4 bcd | 1.11 abc |
| | 45 | 46.8 c | 96.5 cd | 2.10 b | 100.0 a | 17.4 ab | 2.3 d | 79.0 ab | 1.07 c |
| Line | | | | | | | | | |
| Means | | | | | | | | | |
| NWAC103 | | 71.6 a | 110.1 a | 1.57 b | 99.7 a | 17.0 b | 3.8 a | 78.1 b | 1.05 b |
| USDA102 | | 51.6 b | 100.3 b | 2.00 a | 100.0 a | 17.6 a | 3.3 a | 77.9 b | 1.10 a |
| MN | | 46.2 c | 93.6 c | 2.14 a | 99.3 a | 17.1 ab | 2.5 b | 79.0 a | 1.11 a |
| Dietary | | | | | | | | | |
| Protein | | | | | | | | | |
| Means | | | | | | | | | |
| 25 | | 44.8 c | 92.7 c | 2.18 a | 99.0 a | 16.9 a | 3.2 ab | 78.6 a | 1.12 a |
| 35 | | 70.6 a | 107.4 a | 1.56 c | 100.0 a | 17.6 a | 3.6 a | 77.8 b | 1.09 a |
| 45 | | 54.0 b | 103.8 b | 1.97 b | 100.0 a | 17.3 a | 2.9 b | 78.6 a | 1.06 b |

Tables 12 and 13 show that NWAC103 channel catfish fingerlings had significantly better growth, feed conversion and high feed consumption than Kansas juveniles (fingerlings). NWAC103 catfish grew from 7.3 g to 29.1 grams while Kansas catfish grew from 6.3 to 19.6 grams. Survival for both lines was 100%. NWAC103 catfish cultured from juvenile to market size were significantly larger at the end of the growth trial than Kansas catfish (760 vs 531 grams). Males grew faster than females in both lines. Fillet fat increased, fillet moisture decreased and fillet protein remained stable as fish size increased. After adjustment for final size, NWAC103 and Kansas fish did not differ for proximate composition. NWAC103 catfish had shorter, deeper bodies than Kansas catfish, but there was no difference for carcass or fillet yield. Females had higher carcass and fillet yields than males in both lines. Estrogen and testosterone were higher in females than males in both lines, estrogen was higher in NWAC103 females than Kansas females after 180 days, and may be an indication of earlier sexual maturity in NWAC103 females which typically spawn at an earlier age than Kansas catfish females. In both studies, the NWAC103 catfish showed superior performance over the Kansas catfish. Kansas catfish has previously been selected for superior growth characteristics and has been released as an improved line by Auburn University. No significant differences in proximate composition or fillet yield were found, however, serum steroid differences indirectly suggest earlier sexual maturity and improved reproductive performance in NWAC103 catfish.

Table 12 shows comparative growth, feed conversion, and proximate composition of NWAC103 and Kansas channel catfish cultured in experimental tanks. Initial and final weight (g), feed conversion, and survival of NWAC103 and Kansas channel catfish juveniles.

TABLE 12

| | | | | Survival |
|---|---|---|---|---|
| Line | Initial Weight | Final Weight | Feed Conversion | (%) |
| NWAC103 | 7.3 ± 0.1 a | 29.1 ± 0.5 a | 0.87 ± 0.02 a | 100.0 a |
| Kansas | 6.3 ± 0.2 b | 19.6 ± 0.5 b | 1.01 ± 0.02 b | 100.0 a |

Table 13 shows the initial and final weight (g), carcass and fillet yields, proximate compositions, and steroid levels in NWAC103 and Kansas channel catfish cultured in experimental tanks.

TABLE 13

| Variable | NWAC103 | Kansas |
|---|---|---|
| Initial weight (grams) | 30.9 | 28.8 |
| Final weight (grams) | 760 ± 27.9 a | 531 ± 21.3 b |
| Mean carcass yield (%) | 68.4 ± 0.3 a | 68.5 ± 0.2 a |
| Carcass yield females (%) | 70.0 ± 0.3 a | 69.7 ± 0.3 a |
| Carcass yield males (%) | 66.8 ± 0.5 a | 67.3 ± 0.3 a |
| Mean fillet yield (%) | 50.4 ± 0.4 a | 51.0 ± 0.3 |
| Fillet yield males (%) | 48.6 ± 0.7 a | 50.0 ± 0.4 a |
| Fillet yield females (%) | 52.2 ± 0.4 a | 52.0 ± 0.4 a |
| Fillet lipid (%) | 5.9 ± 0.7 a | 5.9 ± 0.7 a |
| Fillet moisture (%) | 76.0 ± 0.6 a | 77.3 ± 0.6 a |
| Fillet protein (%) | 16.7 ± 0.2 a | 16.7 ± 0.2 a |
| Fillet ash (%) | 1.2 ± 0.1 a | 1.3 ± 0.1 a |
| Serum estrogen females (pg/ml) | 1,159.0 ± 54.0 a | 392.8 ± 34.7 b |
| Serum testosterone females (μg/dl) | 2.2 ± 0.2 a | 0.9 ± 0.1 a |
| Serum estrogen males (pg/ml) | 16.8 ± 56.4 a | 30.2 ± 37.4 a |
| Serum testosterone males (μg/dl) | 0.9 ± 0.2 a | 0.7 ± 0.2 a |

Table 14 shows that NWAC103 catfish consumed more feed, gained more weight, and converted feed more efficiently than Norris catfish, regardless of dietary protein levels or feeding rates. Dietary protein level had no significant effect on feed consumption, weight gain, and feed efficiency regardless of fish line or feeding rate. Fish fed to apparent satiation gained more weight, but converted the feed less efficiently than fish fed to approximately ⅔ of the satiation, regardless of fish line and dietary protein level. Survival ranged from 98 to 100%, which did not differ among treatments. Significant interactions between fish line and feeding rate were observed for feed consumption, weight gain, and feed efficiency. Proximate composition of muscle (fillet) samples are to be determined. These data demonstrate that NWAC103 catfish consume more feed, grow faster, and convert feed more efficiency than the Norris catfish. Significant interactions between fish line and feeding rate for feed consumption, weight gain, and feed efficiency indicate a more dramatic separation for these variables for fish fed to apparent satiation than for those fed to approximately ⅔ of satiation. No interaction was observed between fish line and dietary protein level, indicating that the two lines of fish responded to dietary protein concentrations in a similar manner.

Table 14 shows mean feed consumption, weight gain, feed efficiency, and survival of NWAC103 and Norris channel catfish fed to approximate satiation (S) or approximately ⅔ of satiation (or restricted feeding rate, R) with diets containing two dietary protein concentrations for 10 weeks in experimental tanks.

TABLE 14

| | | | | | | |
|---|---|---|---|---|---|---|
| | Dietary | | Feed | | Feed | |
| Fish | protein | Feeding | consumption | Weight gain¹ | efficiency | Survival |
| line | (%) | rate | (g/fish) | (g/fish) | (gain/feed) | (%) |

Individual Treatment Means²

| | | | | | | |
|---|---|---|---|---|---|---|
| NWAC103 | 28 | Satiation | 94.2 | 77.3 | 0.85 | 99.0 |
| Norris | 28 | Satiation | 51.1 | 25.8 | 0.48 | 99.0 |
| NWAC103 | 32 | Satiation | 92.4 | 72.6 | 0.82 | 98.0 |
| Norris | 32 | Satiation | 52.9 | 27.7 | 0.50 | 98.0 |
| NWAC103 | 28 | Restricted | 43.8 | 42.0 | 0.93 | 100.0 |
| Norris | 28 | Restricted | 32.8 | 19.8 | 0.64 | 99.0 |
| NWAC103 | 32 | Restricted | 43.7 | 42.4 | 0.94 | 100.0 |
| Norris | 32 | Restricted | 33.2 | 20.8 | 0.66 | 98.0 |

Pooled Means³

| | | | | | | |
|---|---|---|---|---|---|---|
| NWAC103 | | | 68.5 | 58.6 | 0.88 | 99.3 |
| Norris | | | 42.5 | 23.5 | 0.57 | 98.5 |
| | 28 | | 55.5 | 41.2 | 0.73 | 99.3 |
| | 32 | | 55.5 | 40.9 | 0.73 | 98.5 |
| | | Satiation | 72.6 | 50.8 | 0.66 | 98.5 |
| | | Restricted | 38.4 | 31.3 | 0.79 | 99.3 |

ANOVA

| | | | | |
|---|---|---|---|---|
| Fish line (FS) | P ≦ 0.05 | P ≦ 0.05 | P ≦ 0.05 | NS |
| Dietary protein | NS | NS | NS | NS |
| (DP) | P ≦ 0.05 | P ≦ 0.05 | P ≦ 0.05 | NS |
| Feeding rate (FR) | NS | NS | NS | NS |
| FS × DP | P ≦ 0.05 | P ≦ 0.05 | P ≦ 0.05 | NS |
| FS × FR | NS | NS | NS | NS |
| DP × FR | NS | NS | NS | NS |
| FS × DP × FR | | | | |

¹Mean initial weight was 4.7 and 3.0 g/fish for NWAC103 and Norris catfish
²LSD test was not conducted for individual means because the interaction was not significant.

Table 15 shows that NWAC103 channel catfish consumed similar amounts of feed, but gained more weight, and converted the feed more efficiently than the Stuttgart channel catfish, regardless of dietary protein levels. Regardless of fish line, fish fed the 20% protein diet consumed less feed, gained less weight, and converted the feed less efficiently than fish fed the 28% protein diet. Weight gain and feed efficiency for fish fed the 24% protein diet were equivalent to that of fish fed the 28% protein diet, but higher than fish that of fish fed the 20% protein diet. Feed consumption of fish fed the 24% protein diet was not different from fish fed either 20% or 28% protein diet. There were no differences in survival and visceral fat level among the two lines of fish or among the dietary protein levels. No interactions were observed between fish line and dietary protein level. Proximate composition of muscle (fillet) samples are to be determined. These data demonstrate that NWAC103 channel catfish grow faster and convert feed more efficiently than the Stuttgart catfish. No interactions were observed between fish line and dietary protein level, indicating that the two lines of fish responded to dietary protein concentrations in a similar manner.

Provided in Table 15 below is the mean feed consumption, weight gain, feed efficiency, survival, and visceral fat of NWAC103 and Stuttgart channel catfish fed diets containing three dietary protein concentrations for 8 weeks in experimental tanks.

TABLE 15

| | | | | | | |
|---|---|---|---|---|---|---|
| | Dietary | | | Feed | | Visceral |
| Fish | protein | Feed consumption | Weight gain¹ | efficiency | | fat |
| line | (%) | (g/fish) | (g/fish) | (gain/feed) | Survival (%) | (%) |

Individual Treatment Means²

| | | | | | | |
|---|---|---|---|---|---|---|
| NWAC103 | 20 | 83.9 | 50.3 | 0.60 | 99.0 | 2.90 |
| Stuttgart | 20 | 83.9 | 46.3 | 0.55 | 99.0 | 3.16 |
| NWAC103 | 24 | 87.7 | 61.1 | 0.70 | 96.0 | 2.62 |
| Stuttgart | 24 | 84.9 | 54.9 | 0.64 | 100.0 | 2.93 |
| NWAC103 | 28 | 89.4 | 65.8 | 0.74 | 100.0 | 2.65 |
| Stuttgart | 28 | 87.6 | 59.0 | 0.67 | 99.0 | 2.66 |
| Pooled SEM | | 1.7 | 2.4 | 0.02 | 0.8 | 0.18 |

Pooled Means³

| | | | | | | |
|---|---|---|---|---|---|---|
| NWAC103 | | 87.0 | 59.1 a | 0.68 a | 98.3 | 2.73 |
| Stuttgart | | 85.5 | 53.4 b | 0.62 b | 99.3 | 2.92 |
| | 20 | 83.9 y | 48.3 y | 0.58 y | 99.0 | 3.03 |
| | 24 | 86.3 xy | 58.0 x | 0.67 x | 98.0 | 2.78 |
| | 28 | 88.5 x | 62.4 x | 0.71 x | 99.5 | 2.66 |

ANOVA

| | | | | | |
|---|---|---|---|---|---|
| Fish line | NS | P ≦ 0.05 | P ≦ 0.05 | NS | NS |
| (FS) | NS | P ≦ 0.05 | P ≦ 0.05 | NS | NS |
| Dietary | NS | NS | NS | NS | NS |
| protein (DP) | | | | | |
| FS × DP | | | | | |

¹Mean initial weight was 14.1 g/fish.
²LSD test was not conducted for individual means because the interaction was not significant
³Pooled means within a column and within a factor followed by different letters were different (P ≦ 0.05, LSD test); LSD test was not conducted for pooled means if the main effect was not significant

Table 16 shows that NWAC-103 catfish had a significantly greater weight gain as expressed by the growth index a. Growth hormone treatment and higher temperature significantly increased growth rate. Fish line had the largest effect (a value of NWAC-103 was 33% greater than Norris), followed by temperature (28% difference in a values), followed by growth hormone treatment (20% difference in a values). The interaction between growth hormone treatment and temperature was also significant (P<0.02), showing that an the difference between growth at 22 and 260C. was greater when the fish were not treated with growth hormone. IGF-1 levels were significantly higher in NWAC103 catfish, in fish injected with GH, and in fish at warmer temperatures. The line by injection treatment interaction was significant because NWAC103 catfish had greater IGF-1 plasma levels in response to rbGH injection than Norris catfish. The growth of NWAC103 catfish was superior to the growth of Norris channel catfish. The use of the growth index enables comparison of growth of fish that began at different sizes. In addition to line differences, it was shown that growth of both catfish can be improved by growth hormone treatment, and IGF-1 levels correlated with growth rate even at temperatures well below the 28 to 300C. range considered optimal for channel catfish growth. This finding is especially relevant because of its potential to increase the duration of the growing season, through GH treatment or potentially through growth hormone transgenic animals. Data on feed intake, feed efficiency, and proximate composition of the fish from this study was collected and is currently being analyzed.

Shown below in Table 16 is the least square means for the growth index (a), feed consumption, feed efficiency, IGF-1 levels, and proximate analysis of NWAC103 catfish and Norris catfish at two different culture temperatures and receiving growth hormone or saline injection treatments in experimental tanks.

TABLE 16

| | Temp. | | Growth Index | Feed | Feed | IGF-1 | Percent | Percent | Percent |
|---|---|---|---|---|---|---|---|---|---|
| Line | (° C.) | Injection | (a) | Consump. | Efficiency | (ng/ml) | Moisture | Fat | Protein |
| Norris | 21.7 | GH | 1.77 ± 0.10 | 1.79 ± 0.09^{b} | ^{c} | 8.09 ± 72^{b} | 76.7 ± 0.8^{a} | 4.6 ± 0.2^{b} | 17.4 ± 0.6^{a} |
| | 21.7 | Saline | 1.17 ± 0.15 | 1.51 ± 0.22^{a} | 0.45 ± 0.02^{a} | 4.19 ± 0.36^{a} | 76.5 ± 1.4^{a} | 3.3 ± 0.8^{ab} | 18.7 ± 0.6^{ab} |
| | 26.0 | GH | 2.19 ± 0.20 | 2.05 ± 0.21^{cd} | 0.99 ± 0.08^{d} | 8.81 ± 0.70^{b} | 75.3 ± 0.3^{a} | 5.0 ± 0.5^{ab} | 18.4 ± 0.8^{ab} |
| | 26.0 | Saline | 1.98 ± 0.12 | 1.68 ± 0.18^{ab} | 0.99 ± 0.05^{d} | 5.39 ± 0.28^{a} | 76.7 ± 0.3^{a} | 3.1 ± 0.2^{a} | 19.0 ± 0.2^{b} |
| NWAC103 | 21.7 | GH | 2.32 ± 0.05 | 2.21 ± 0.08^{dc} | 0.78 ± 0.05^{c} | 12.07 ± 0.92^{c} | 74.4 ± 1.4^{a} | 7.3 ± 0.9^{b} | 16.9 ± 0.3^{a} |
| | 21.7 | Saline | 1.95 ± 0.08 | 1.86 ± 0.03^{bc} | 0.63 ± 0.04^{b} | 5.12 ± 0.24^{a} | 75.3 ± 0.6^{a} | 5.2 ± 0.5^{a} | 18.2 ± 0.5^{b} |
| | 26.0 | GH | 2.68 ± 0.11 | 2.38 ± 0.03^{c} | 0.98 ± 0.06^{d} | 12.03 ± 1.15^{c} | 74.8 ± 1.6^{a} | 6.9 ± 0.6^{ab} | 17.1 ± 0.8^{ab} |
| | 26.0 | Saline | 2.46 ± 0.06 | 2.06 ± 0.10^{cd} | 0.94 ± 0.02^{d} | 7.76 ± 0.45^{b} | 74.7 ± 1.1^{a} | 6.4 ± 1.1^{ab} | 17.6 ± 0.5^{ab} |

Tables 17 and 18 show that the growth index a, of restricted (1% body weight) ration groups was higher (P<0.001, n=5) for NWAC103 catfish than for Norris catfish, indicating a faster growth rate. Likewise, the NWAC103 catfish on the satiation feeding regime had significantly faster growth rates than Norris catfish fed to satiation (P<0.001, n=5). Under both feeding regimes NWAC103 catfish outperformed Norris catfish in feed conversion ratio. At the 1% body weight feeding rate, Norris catfish required 1.8 times as much food to gain a unit of weight as NWAC103 catfish. The restricted ration groups for both lines had higher feed conversion ratios than the satiation fed groups. Weight gain between 20 Jan. 1998, and 11 Feb. 1998 was higher in NWAC103 catfish than Norris catfish. In addition to line effects, feeding treatment had a marginally significant effect on weight gain (P=0.055). The 2 day fast at the end of the 3 week experiment caused a mean decline of 2.7 g fish-1, or 27% lower body weight gain compared to fish not experiencing a fast. Food consumption was greater in NWAC103 catfish than in Norris catfish (2.90″0.25 g vs. 1.18″0.15 g; P<0.001). Weight of the individual fish had a significant effect on the amount of food consumed (r=0.47, P<0.001), and was therefore retained in the ANCOVA model as a covariate. The effect of fasting on food consumption was not significant overall (P>0.50); however, the line by feeding treatment interaction was highly significant (P<0.001). The fasted Norris fish ate more (1.60″0.21 g) than the fed Norris fish (0.75″0.18 g; P<0.005), whereas the fed NWAC103 fish ate more (3.54″0.35 g) than the fasted NWAC103 fish (2.28″0.31 g; P<0.005). When the fasting treatment was repeated on NWAC103 fish in experiment 3, a consistent result was obtained. Food consumption did not differ between fish fasted for 4, 2 or 0 days. NWAC103 catfish outperformed Norris catfish for growth and food conversion ratio under both restricted and satiation feeding regimes, as indicated by a values, a growth rate index. The food conversion ratios for these treatment groups showed that NWAC103 catfish were more efficient in feed utilization, overall. The higher food conversion ratios observed for the fish on restricted ration in both lines demonstrated that the 1% W ration was quite restrictive for both lines. Food intake measurements in experiment 2 showed that the NWAC-103 catfish ate nearly twice the amount of food as Norris catfish, and also responded differently to a 2 day fast. Although this work does not permit identification of the physiological mechanisms regulating food intake, it is clear that the 2 lines studied under common environmental conditions responded differently to food following a short deprivation. These two lines, with differences in food intake and food intake regulation, provide a model system for further work on the mechanisms of food intake regulation and may help to identify specific traits for genetic selection to improve food intake in catfish.

Table 17 provides Intercept (a) values from the growth rate equation: loge Gw=a-0.371 loge Wm, and food conversion ratios for NWAC103 and Norris catfish on satiation and 1% body weight feeding regimes. Values in rows with different superscripts are significant differences (P<0.05).

TABLE 17

| | Initial weight | End weight | Weight gain¹ | Consumption (g) | Consumption (% W) |
|---|---|---|---|---|---|
| Fed | | | | | |
| Norris (20) | 59.4 ± 1.0^{a} | 66.2 ± 2.4^{a} | 7.1 ± 0.1^{b} | 0.75 ± 0.18^{a} | 1.09 ± 0.24^{a} |
| NWAC-103 (17) | 59.9 ± 1.1^{a} | 72.8 ± 3.1^{b} | 13.1 ± 1.7^{d} | 3.54 ± 0.35^{c} | 4.76 ± 0.39^{c} |
| Fasted | | | | | |
| Norris (20) | 59.4 ± 1.0^{a} | 64.1 ± 1.3^{a} | 5.0 ± 0.8^{a} | 1.60 ± 0.21^{b} | 2.47 ± 0.31^{b} |
| NWAC-103 (18) | 59.9 ± 1.1^{a} | 69.1 ± 3.5^{a} | 9.9 ± 0.3^{c} | 2.28 ± 0.31^{b} | 3.36 ± 0.54^{b} |

¹Weight gain was based on tank mean weights.

Table 18 shows initial weights, end weights, weight gain, weight of food consumed, and percentage of body weight consumed by Norris and NWAC-103 catfish under fed and fasted conditions. The numbers in parentheses indicate sample sizes. In columns, values with different superscripts indicate significant differences (P<0.05).

TABLE 18

| | Initial weight | End weight | Weight gain¹ | Consumption (g) | Consumption (% W) |
|---|---|---|---|---|---|
| Fed | | | | | |
| Norris (20) | 59.4 ± 1.0^{a} | 66.2 ± 2.4^{a} | 7.1 ± 0.1^{b} | 0.75 ± 0.18^{a} | 1.09 ± 0.24^{a} |
| NWAC-103 (17) | 59.9 ± 1.1^{a} | 72.8 ± 3.1^{b} | 13.1 ± 1.7^{d} | 3.54 ± 0.35^{c} | 4.76 ± 0.39^{c} |
| Fasted | | | | | |
| Norris (20) | 59.4 ± 1.0^{a} | 64.1 ± 1.3^{a} | 5.0 ± 0.8^{a} | 1.60 ± 0.21^{b} | 2.47 ± 0.31^{b} |
| NWAC-103 (18) | 59.9 ± 1.1^{a} | 69.1 ± 3.5^{a} | 9.9 ± 0.3^{c} | 2.28 ± 0.31^{b} | 3.36 ± 0.54^{b} |

¹Weight gain was based on tank mean weights

Table 19 shows initial weights, end weights, weight gain, weight of food consumed, and percentage of body weight consumed by NWAC-103 catfish under fed, 2 days (2 d) fasted and 4 days (4 d) fasted conditions. The numbers in parentheses indicate sample sizes. In columns, values with different superscripts indicate significant differences.

TABLE 19

| Treatment | Initial weight | End weight | Weight gain | Consumption (g) | Consumption (% W) |
|---|---|---|---|---|---|
| Fed (10) | 71.2 ± 3.7^{a} | 78.4 ± 3.4^{a} | 7.1 ± 1.5^{b} | 3.92 ± 0.87^{a} | 5.01 ± 0.23^{a} |
| 2 d fast (9) | 76.7 ± 2.8^{a} | 76.7 ± 3.0^{a} | 0.0 ± 2.2^{a} | 3.43 ± 1.09^{a} | 4.46 ± 0.33^{a} |
| 4 d fast (10) | 73.9 ± 2.8^{a} | 75.8 ± 3.1^{a} | 1.9 ± 1.1^{a} | 3.77 ± 0.78^{a} | 5.00 ± 0.19^{a} |

Table 20 shows that catfish line and feeding had a significant affect on catfish mortality following exposure to Edwardsiella ictaluri. NWAC103 catfish had lower mortality than Norris catfish when starved (20.7 vs 26.1%) or fed to satiation (44.6 vs 57.5%). Feed consumption following bacterial exposure was reduced in Norris catfish compared to NWAC103 catfish. Over the entire challenge period (28 days), NWAC103 catfish consumed over 3 times more feed than Norris catfish (366 vs 102 grams). In this experimental challenge study, NWAC103 catfish had significantly lower mortality than Norris catfish, however, results demonstrate that feeding practice or feeding rate has more impact on mortality than the fish line. The least squares mean mortality for fed fish was 51.1% versus 23.4% for non-fed fish representing a 27% difference in mortality. The difference in mortality between the lines was 9.1%. No line*feeding practice interaction was found.

Provided in Table 20 is the mean (+SE) mortality and feed consumption of NWAC103 and Norris channel catfish following experimental challenge with Edwardsiella ictaluri in experimental aquaria.

TABLE 20

| Variable | NWAC103 | Norris |
|---|---|---|
| Mortality (%) - fed to satiation | 44.6 ± 4.0 | 57.5 ± 4.6 |
| Mortality (%) - non fed | 20.7 ± 3.1 | 26.1 ± 3.8 |
| Feed consumption (grams) | 366 | 102 |
| Line mean ± SE (lsmean) | 32.7 ± 0.03 | 41.8 ± 0.03 |

Table 21 shows that purebred USDA102 catfish and crosses among USDA102 and other lines generally had higher survival and lower antibody production 30 d after challenge with live, virulent Edwardsiella ictaluri relative to Norris and NWAC103 catfish and their crosses. There were no differences among genetic groups for antibody response to formalin-killed Edwardsiella ictaluri. USDA102 catfish contributed additive and dominance effects for increased survival and lower antibody level after live challenge. Results indicate that differences exist among genetic groups for survival and antibody production after live Edwardsiella ictaluri challenge, but these differences were not related to differences among genetic groups for antibody response to killed Edwardsiella ictaluri. USDA102 catfish contributed favorable genetic effects for survival after challenge with live Edwardsiella ictaluri. However, interactions among genotype, Edwardsiella ictaluri resistance, and food intake were not considered. Previous and subsequent research described in this report has shown that increased food intake is positively correlated with Edwardsiella ictaluri mortalities and the NWAC103 catfish is an aggressive feeder. All fish were fed to satiation following the Edwardsiella ictaluri challenge in this study, and it was subjectively observed that NWAC103 catfish were actively feeding relative to the other lines, but food intake was not quantified. Therefore, it is possible the genetic effects for Edwardsiella ictaluri resistance observed in this study reflect genetic effects for food intake during the post-challenge period. It will be important in future experimental ESC challenges and in management of commercial stocks to consider interactions between food intake and Edwardsiella ictaluri resistance.

Shown in Table 21 is the least square means (+average standard errors) for survival after challenge with live Edwardsiella ictaluri, antibody level after challenge with live Edwardsiella ictaluri and antibody level after injection with formalin killed Edwardsiella ictaluri.

TABLE 21

| | Live | Live | Killed |
|---|---|---|---|
| | Challenge | Challenge Ab | Challenge |
| Genotype | Survival | Level | Ab Level |
| (Female × Male) | (%) | (OD) | (OD) |
| USDA102 × USDA102 | 89.7 | 0.169 | 0.180 |
| USDA102 × NWAC103 | 79.0 | 0.160 | 0.191 |
| USDA102 × Norris | 83.9 | 0.176 | 0.194 |
| NWAC103 × USDA102 | 92.8 | 0.139 | 0.198 |
| Norris × USDA102 | 88.7 | 0.192 | 0.210 |
| Norris × Norris | 65.2 | 0.198 | 0.200 |
| NWAC103 × NWAC103 | 51.1 | 0.187 | 0.211 |
| Norris × NWAC103 | 53.6 | 0.232 | 0.195 |
| NWAC103 × Norris | 70.8 | 0.227 | 0.201 |
| Average | 10.5 | 0.022 | 0.025 |
| Standard | | | |
| Error | | | |

Table 22 shows that initial stocking size, initial weight did not have a significant effect on harvest weight. NWAC103 catfish outgrew all other groups and were significantly larger at harvest. Significant variation was found in survival with the NWAC103, USDA102, albino, and Norris x blue hybrid having the highest survival followed by Kansas and Mississippi-normal catfish, and the Norris catfish with lowest survival. Significant variation also occurred in trimmed fillet yield with Norris x blue hybrids and NWAC103 highest followed by Kansas and Norris, with USDA102, albino, and Mississippi-normals having the lowest dressout percentage. Significant differences in harvest weight were found only for NWAC103 catfish. All other catfish were not significantly different. Survival ranged from 43.5% for Norris catfish to 95.0% for the Norris x blue hybrids, some significant variation in survival was found between catfish groups. Significant variation in trim fillet yield was found between catfish groups with hybrid catfish having the highest trim and adjusted trim fillet yield of all groups. NWAC103 channel catfish ranked second in trim fillet yield and were significantly lower only when the trim fillet yield was adjusted for size. Growth of six channel catfish lines and one channel x blue hybrid cultured communally in earthen ponds. at the USDA/ARS Catfish Genetics Research Unit, Stoneville, Miss.

Shown in Table 22 is growth of six channel catfish lines and one channel x blue hybrid cultured communally in earthen ponds. at the USDA/ARS Catfish Genetics Research Unit, Stoneville, Miss.

TABLE 22

| | Stocking | Harvest | Survival | Trim Fillet | Adjusted Trim |
|---|---|---|---|---|---|
| Line | Weight (g) | Weight (g) | (%) | Yield (%) | Fillet Yield (%) |
| Albino | 129.6 ± 1.6 a | 500.8 ± 45.8 b | 81.0 ± 5.6 ab | 41.9 ± 0.4 cd | 41.9 ± 0.5 bc |
| Kansas | 102.7 ± 1.9 b | 446.9 ± 81.3 b | 58.8 ± 10.7 b | 43.4 ± 0.4 bc | 43.5 ± 0.5 b |
| MN | 118.0 ± 5.7 a | 468.6 ± 70.3 b | 64.0 ± 11.4 b | 41.9 ± 0.6 cd | 42.0 ± 0.5 bc |
| Norris | 85.1 ± 4.1 cd | 411.4 ± 21.3 b | 43.5 ± 0.6 c | 43.5 ± 0.6 bc | 43.9 ± 0.6 b |
| Norris × Blue | 80.0 ± 3.6 d | 536.1 ± 27.7 b | 95.0 ± 8.4 a | 46.1 ± 0.6 a | 46.1 ± 0.5 a |
| USDA102 | 94.1 ± 3.1 bc | 413.4 ± 23.6 b | 78.5 ± 5.3 ab | 41.3 ± 0.5 d | 41.5 ± 0.5 c |
| NWAC103 | 117.2 ± 8.0 a | 859.6 ± 138.0 a | 82.8 ± 13.6 ab | 44.8 ± 0.6 ab | 44.1 ± 0.8 b |

Table 23 shows there was significant variation in stocking weight, final weight, and relative growth rates and post-harvest antibody levels. NWAC103 line channel catfish had the largest initial size, final weight, and instantaneous growth rates, however, there were no significant differences in survival or relative growth rates. Antibody levels at harvest were positive (optical density=0.60+0.03 in communal ponds) for all groups of catfish showing successful exposure of fish to Edwardsiella ictaluri. Survival in the communally stocked ponds ranged from 83.3-93.3%. This study was designed to simulate conditions of high stocking rate and pathogen exposure that juvenile fish might be exposed to in a commercial pond environment. Overall growth rates were consistent with other studies and observations showing the channel x blue hybrid and NWAC103 lines to be the fastest growing catfish groups. No significant differences in survival were found in this study.

Table 23 shows growth and survival data of seven catfish lines and one channel x blue hybrid stocked communally in earthen ponds at the USDA/ARS Catfish Genetics Research Unit, Stoneville, Miss.

TABLE 23

| | Stocking | | Relative | | |
|---|---|---|---|---|---|
| Line | Weight | Harvest Weight | Growth (%) | Survival (%) | Antibody Level |
| NWAC103 | 145.3 ± 6.2 a | 292.2 ± 49.6 a | 101.1 ± 34.2 a | 90.0 ± 2.0 a | 0.75 ± 0.06 b |
| USDA102 | 88.1 ± 3.7 b | 157.8 ± 37.9 b | 79.1 ± 43.1 a | 91.7 ± 0.6 a | 0.58 ± 0.06 bc |
| Albino | 82.7 ± 2.7 d | 123.6 ± 11.7 bc | 49.5 ± 14.1 a | 90.1 ± 1.8 a | 0.53 ± 0.07 c |
| Marion-SR | 85.3 ± 2.7 c | 123.7 ± 8.5 bc | 45.0 ± 9.9 a | 86.5 ± 1.3 a | 0.75 ± 0.07 b |
| MN | 70.3 ± 0.7 e | 113.5 ± 9.0 bc | 61.5 ± 12.7 a | 92.7 ± 0.6 a | 0.49 ± 0.05 c |
| Norris | 55.3 ± 0.8 f | 84.6 ± 6.6 bc | 53.0 ± 11.9 a | 84.3 ± 2.4 a | 0.95 ± 0.06 a |
| Norris × blue | 70.3 ± 1.9 e | 137.8 ± 12.1 bc | 96.0 ± 17.2 a | 93.3 ± 1.1 a | 0.22 ± 0.03 d |
| Marion | 44.9 ± 0.7 g | 71.2 ± 1.6 c | 58.6 ± 3.6 a | 86.9 ± 1.1 a | 0.62 ± 0.08 bc |

Table 24 shows that over the 2 months of this study, growth of the fish as assessed by the growth index a was significantly slower in Norris catfish, than in either NWAC103 or Kansas catfish. Growth of Kansas and NWAC-103 catfish was equivalent. Feed intake followed a similar pattern. Feed intake of the Norris catfish was significantly lower than in Kansas and NWAC103 fish, whereas the intake of Kansas and NWAC103 catfish was the same. Survival over the course of this study was 89% overall. There were no differences in survival by pond or line. Over the 4 days of monitoring feed intake, there was some variation in catfish performance. While feed intake of Kansas and NWAC103 catfish was generally superior to Norris intake, at the 11-13 August sampling feed intake by NWAC103 fish was low. Growth of the NWAC103 line through this interval was slower than in other intervals. Comparisons of feed intake and growth rate in this study were complicated by differences in initial sizes of the fish. Nevertheless, growth and feed intake of the NWAC103 and Kansas catfish were superior to the Norris catfish. Low feed intake corresponded to poor growth in NWAC103 over the second interval measured. Considerably improved intake by NWAC103 catfish in the final sampling period had not yet been expressed in greater growth rate by the end of the trial. Repeated sampling of fish in ponds may have contributed to variation in feed intake over the course of the study.

Provided in Table 24 are the mean weights (“S. E.) of Norris, Kansas, and NWAC103 catfish at each sampling period (n=106 to 230), and index of growth rate (a value) communally stocked in replicated earthen ponds in 1998 at the USDA/ARS Catfish Genetics Research Unit, Stoneville, Miss.

TABLE 24

| Line | LineJul 27-29 | Aug 11-13 | Sep 1-3 | Sep 15-17 | a |
|---|---|---|---|---|---|
| Norris | 36.0 ± 1.2 | 46.5 ± 1.6 | 72.2 ± 2.6 | 85.9 ± 3.9 | 2.11 ± 0.08^{a} |
| Kansas | 44.2 ± 1.1 | 66.7 ± 1.5 | 104.7 ± 2.5 | 119.7 ± 3.4 | 2.52 ± 0.05^{b} |
| NWAC-103 | 100.6 ± 3.0 | 119.7 ± 3.6 | 188.7 ± 6.0 | 224.8 ± 7.8 | 2.52 ± 0.06^{b} |

Tables 25-31 provide growth, survival and yield data for NWAC103 catfish as compared to other lines of channel catfish by specific research ponds.

USDA/ARS Catfish Genetics Research Unit Ponds:

During the 1997 growing season NWAC103 channel catfish were compared with Norris and Kansas catfish. Harvest weight and yield were significantly higher in NWAC103 catfish than the other two lines. Feed conversion, survival and dressout percentage were not significantly different. During the 1998 growing season, NWAC103 catfish were compared to Kansas catfish and blue x channel catfish hybrids. Harvest weight and weight gain were significantly higher in NWAC103 catfish than the other two catfish groups. Feed conversion and yield were not significantly different. Survival was significantly lower in NWAC103 fish because of fish losses in two ponds from low oxygen levels. These losses reduced the overall yield in NWAC103 fish to levels significantly different from the other two groups and probably caused the poorer feed conversion even though it was not significantly different. Dressout percentage in NWAC103 and Kansas catfish was not significantly different, however, hybrids had significantly higher fillet yield.

MAFES Research Ponds:

During the 1996 growing season, NWAC103 channel catfish fry were stocked into earthen ponds and compared with Norris and Kansas catfish. NWAC103 fingerlings consumed more feed, were larger, and had a greater yield than the other two lines. Survival was not significantly different. During the 1997 growing season, the fingerlings produced in earthen ponds were restocked for growout to marketable sizes. At the end of the growing season, NWAC103 catfish had a significantly higher yield than either Kansas or Norris catfish. Weight gain was higher than Norris catfish, but not Kansas catfish. Feed conversion was the same as Norris catfish, but significantly worse than Kansas catfish. No difference was found for survival. During the 1998 growing season, NWAC103 catfish had a significantly higher harvest weight, feed consumption, and net yield than Kansas catfish. The feed conversion and survival were significantly lower than Kansas catfish. No difference was found for dressout percentage. NWAC103 catfish also had significantly higher harvest weight, weight gain and yield than Mississippi normal catfish when fed three different protein diets during the 1999 growing season. There was no difference in feed conversion efficiency, survival, or fillet yield.

USDA/ARS Aquaculture Systems Research Unit Ponds:

During the 1998 growing season, NWAC103 catfish were compared with Kansas catfish. The study was terminated in the spring of 1999, and fish in all ponds experienced disease mortalities from winterkill, proliferative gill disease and enteric septicemia during the winter and spring of 1999. Significantly lower survival and feed conversion were found for NWAC103 catfish. No differences were found for harvest weight, weight gain, feed consumption or dressout percentage.

Results demonstrated that the NWAC103 catfish generally performed better for growth and yield characteristics than Kansas and Norris channel catfish and hybrid catfish in ponds at the USDA/ARS Catfish Genetics Research Unit and in MAFES ponds. No differences were found between channel catfish lines for dressout percentage at any of the locations, however, hybrid blue x channel catfish always had a better dressout percentage. Results for feed conversion and survival were more variable and are directly related; lower survival generally results in poor feed conversion. In one pond study feed conversion and survival were better for NWAC103 catfish, in one study there were no differences, in one study feed conversion was not different, but survival was worse, in one other study survival was not different, but feed conversion was worse, and in two studies the NWAC103 catfish were worse for both feed conversion and survival than the compared line. The poor survival found in studies at UAPB may have been related to low pond chloride levels. Fish stocked in an ongoing study (1999-2000) initially had low level mortality unrelated to disease until pond chloride levels were raised to 100 ppm. It can be concluded that pond studies generally show more variability than aquarium or tank studies, and because of space and cost limitations, fewer replicates (sometimes only 3) are often utilized in pond experiments. The greater variability often results in a lack of statistical significance (p<0.05) in measured traits.

Table 25 shows the growth, survival, feed conversion, and yield (mean+SE) of three catfish groups cultured in replicate 0.1-acre earthen ponds during 1997 at the USDA/ARS Catfish Genetics Research Unit, Stoneville, Miss.

TABLE 25

| | | | Blue × Channel |
|---|---|---|---|
| Variable | NWAC103 | Norris | Hybrid |
| Stock weight (g) | 57 ± 2a | 27 ± 1b | 46 ± 3a |
| Harvest weight (g) | 655 ± 21a | 389 ± 43c | 503 ± 30b |
| Feed conversion | 1.82 ± 0.07a | 1.78 ± 0.06a | 1.85 ± 0.05a |
| Yield (lbs/acre) | 6125 ± 197a | 3640 ± 406b | 4701 ± 281b |
| Survival (%) | 94.6 ± 1.5a | 89.8 ± 1.8a | 91.5 ± 2.0a |
| Dressout percent | 45.2a | 44.7a | 48.0b |

Table 26 shows pond (mean+SE) growth, survival, feed conversion, and yield of three catfish groups cultured in replicate 0.1-acre earthen ponds during 1998 at the USDA/ARS Catfish Genetics Research Unit, Stoneville, Miss.

TABLE 26

| | | | Blue × Channel |
|---|---|---|---|
| Variable | NWAC103 | Kansas | Hybrid |
| Stock weight (lb) | 0.12 ± .01a | 0.08 ± 0.01b | 0.07 ± 0.01b |
| Harvest weight (lb) | 1.76 ± .09a | 1.24 ± .03b | 1.08 ± .06b |
| Weight gain (lb) | 1.63 ± .09a | 1.16 ± .01b | 1.02 ± .06b |
| Feed conversion | 1.74 ± .02a | 1.62 ± .05a | 1.64 ± .06a |
| Yield (lbs/acre) | 7,786 ± 294a | 7,221 ± 196a | 6,688 ± 317a |
| Survival (%) | 71.2 ± 5.1a | 91.4 ± 0.9b | 94.3 ± 3.1b |
| Dressout percent | 45.2a | 44.7a | 48.0b |

Table 27 shows growth, feed consumption, feed conversion, yield, and survival data of 3 lines of channel catfish fry cultured to fingerlings in replicate 0.1-acre earthen ponds during 1996 at the Thad Cochran National Warmwater Aquaculture Center by MAFES scientists.

TABLE 27

| | Feed | Mean Weight | | Feed | Survival |
|---|---|---|---|---|---|
| Line | Consumption | (lbs/1000) | Yield | Conversion | (%) |
| NWAC103 | 276 ± 11a | 65.8 ± 4.2a | 259.0 ± 7.9a | 1.06 ± 0.02a | 79.0 ± 2.7a |
| Norris | 154 ± 23c | 48.2 ± 6.0b | 134.0 ± 19.2c | 1.15 ± 0.04a | 57.0 ± 9.2a |
| Kansas | 246 ± 4b | 53.9 ± 2.1b | 203.0 ± 9.6v | 1.22 ± 0.04a | 75.0 ± 1.5a |

Table 28 shows growth, feed consumption, feed conversion, yield, and survival data of 3 lines of channel catfish fingerlings cultured to marketable size in replicate 0.1-acre earthen ponds during the 1997 growing season at the Thad Cochran National Warmwater Aquaculture Center by MAFES scientists.

TABLE 28

| | Weight gain | Feed | | Yield |
|---|---|---|---|---|
| Line | (lbs) | Conversion | Survival (%) | (lbs/acre) |
| NWAC103 | 0.95 ± 0.05a | 1.60 ± 0.02a | 91.4 ± 6.1a | 6414 ± 167a |
| Kansas | 0.88 ± 0.03ab | 1.51 ± 0.01b | 76.3 ± 5.0a | 5033 ± 318b |
| Norris | 0.77 ± 0.03b | 1.60 ± 0.02a | 83.2 ± 7.1a | 4775 ± 260b |

Table 29 shows growth, feed consumption, feed conversion, yield, and survival data of 2 lines of channel catfish fingerlings cultured to marketable size in replicate 0.1-acre earthen ponds during the 1998 growing season at the Thad Cochran National Warmwater Aquaculture Center by MAFES scientists

TABLE 29

| | | | | | Feed | |
|---|---|---|---|---|---|---|
| | Harvest | | | | Con- | |
| | weight | | Survival | Net Yield | sumed | Dressout |
| Line | (lbs) | FCR | (%) | (lbs/acre) | (lb/fish) | Percent |
| NWAC103 | 1.61a | 1.78a | 89.2a | 9,266a | 2.63a | 44.3a |
| Kansas | 1.26b | 1.52b | 98.3b | 8,203b | 1.82b | 43.6a |

Table 30 shows growth, feed consumption, feed conversion, yield, and survival data of 2 lines of channel catfish fingerlings cultured to marketable size in replicate 0.1-acre earthen ponds during the 1999 growing season at the Thad Cochran National Warmwater Aquaculture Center by MAFES scientists

TABLE 30

| | | | | | Feed | |
|---|---|---|---|---|---|---|
| | Harvest | | | | Con- | Dress- |
| | weight | | Survival | Net Yield | sumed | out |
| Line | (lbs) | FCR | (%) | (lbs/acre) | (lb/fish) | Percent |
| NWAC103 | 0.96 a | 1.65 a | 96.8 a | 10,641 a | 1.46 a | 45.4 a |
| MN | 0.93 b | 1.60 a | 94.9 a | 9,374 b | 1.32 b | 44.7 a |

Table 31 shows growth, survival, feed conversion, and yield (mean+SE) of two catfish groups cultured in replicate 0.25-acre earthen ponds during 1998 at the USDA/ARS Aquaculture Systems Research Unit, Pine Bluff, Ark.

TABLE 31

| Variable | NWAC103 | Kansas |
|---|---|---|
| Stock weight (lb) | 0.16 ± .01a | 0.08 ± 0.01b |
| Harvest weight (lb) | 1.56 ± .08a | 1.33 ± .06a |
| Weight gain (lb) | 1.40 ± .08a | 1.25 ± .06a |
| Feed conversion | 2.65 ± .03a | 1.79 ± .09b |
| Yield (lbs/acre) | 5,300 ± 768a | 8,596 ± 688a |
| Survival (%) | 48.5 ± 4.3a | 92.3 ± 6.3b |
| Feed Consumed | 3,340+ ± 135a | 3,818 ± 166a |
| Dressout percent | 42.5 ± 1.0a | 42.11 ± 0.3a |

Tables 32 and 33 show that compared to Norris catfish fed 32% protein diet, NWAC103 catfish fed 32% protein diet were larger at stocking (69.1 g vs. 49.7 g, P=0.02), larger at harvest (531 g vs. 352 g, P=0.009), had faster growth rate (3.2 g/day vs. 2.0 g/day, P=0.008), higher percent growth (755% vs. 666%, P=0.09), and better feed conversion (1.63 vs. 1.78, P=0.08). Survival of NWAC103 and Norris catfish was not different (87.3 vs. 88.9%, P=0.35). Males were larger than females at harvest in both lines (473 g vs. 410 g, P=0.0003). Processing traits were not different between NWAC103 and Norris catfish fed 32% protein diet. Compared to males, females had lower gutted yield (89.0% vs. 90.6%, P=0.0001), higher headed-gutted yield (66.7% vs. 65.7%, P=0.0001), higher shank-fillet yield (37.5% vs. 36.0%, p=0.0003), and lower nugget yield (8.4% vs. 8.8%, P=0.008) when values were averaged over lines. Line*sex interactions were not significant for any traits measured. NWAC103 catfish fed 22% or 32% protein diets were similar for harvest weight (507 g vs. 532 g, P=0.26), growth rate (3.0 g/day vs. 3.2 g/day, P=0.33), percent growth (731% vs. 755%, P=0.65), FCR (1.66 vs. 1.63, P=0.60), and survival (89.7 vs. 87.3, P=0.16). NWAC103 males were larger at harvest than NWAC103 females on both 22% and 32% protein diets (557.1 g vs. 472.2 g, P 0.0001). Compared to NWAC103 catfish fed 22% protein diet, NWAC103 fish fed 32% protein had higher headed-gutted yield (66.7% vs. 65.1%, P=0.02) and higher shank fillet yield (37.5% vs. 36.2%, P=0.01). Gutted yield and nugget yield were not different between fish fed 22% and 32% protein diets. Compared to NWAC103 males, NWAC103 females had lower gutted yield (88.8% vs. 90.2%, P=0.0001), higher headed-gutted yield (66.1% vs 65.2, P=0.0001), higher shank fillet yield (37.4% vs. 36.0%, P=0.0002), and lower nugget yield (8.4% vs 8.7%, P=0.004) when averaged over diets. Diet*sex interaction were not significant for any traits measured. Results indicate that culture of NWAC103 catfish would shorten the production cycle and lower feed costs relative to Norris catfish and producers would benefit by growing NWAC103 catfish or similar improved catfish germplasm. There were no differences in processing traits among NWAC103 and Norris catfish. Our results confirm other reports of faster growth but lower shank fillet yield in males compared to females and that differences between sexes for growth and fillet yield are consistent across lines. NWAC103 catfish fed 22% and 32% protein diets had similar growth, feed conversion (FCR), and survival indicating that lowering production costs through lowering dietary protein levels is possible. However, shank fillet yield was about 1.25% lower for fish fed 22% protein diet than for fish fed 32% protein diet and the effects of reducing dietary protein to this extent (10%) on fillet yield need to be considered in economic evaluations. Ultimately the catfish farming industry will benefit from use of improved germplasm grown in production environments optimized to maximize profits. However, because variables that influence profits such as feed prices, fish prices, fillet yield, and fillet prices fluctuate across time it is difficult to determine what the most profitable combination of improved germplasm and production environment will be. NWAC103 catfish outperformed Norris catfish (a line currently used by the industry) for growth traits and the NWAC103 catfish exhibited similar growth, FCR, and survival when fed 22 or 32% protein diets. Commercial use of NWAC103 catfish should benefit the catfish farming industry and the superior growth performance of NWAC103 catfish should be retained on lower protein diets.

Table 32 shows growth traits, feed conversion ratio (FCR), and survival (mean+S.E) for of NWAC103 channel catfish fed 22% and 32% dietary protein and Norris catfish fed 32% dietary protein during 1999 at the USDA/ARS Catfish Genetics Research Unit, Stoneville, Miss.

TABLE 32

| | NWAC103 vs Norris | | | | 22% Protein Diet vs. 32% Protein | | | |
|---|---|---|---|---|---|---|---|---|
| | 32% Protein Diet | | | | Diet NWAC 103 | | | |
| | NWAC103 | Norris | S.E. | Effects* | 32% | 22% | S.E. | Effects* |
| Stocking | 69.1 | 49.7 | 3.1 | Line | 69.1 | 65.0 | 3.0 | — |
| Harvest | 571.2 males | 376.3 males | 27.7 | Line, Sex | 571.2 males | 542.9 males | 12.1 | Sex |
| Weight (g) | 492.5 females | 491.7 females | | | | 472.3 females | | |
| Growth | 3.2 | 2.0 | 0.17 | Line | 3.2 | 3.0 | 0.15 | — |
| Growth (%) | 755 | 666 | 36 | Line | 755 | 732 | 32 | — |
| FCR | 1.63 | 1.78 | 0.06 | Line | 1.63 | 1.66 | 0.03 | — |
| Survival | 87.3 | 88.9 | 1.2 | — | 87.3 | 89.7 | 1.1 | — |

*Indicates effect is significant at P < 0.10.

Table 33 shows processing yield traits (means+S.E) for NWAC103 catfish fed 22% and 32% protein diets and Norris catfish fed 32% dietary protein during 1999 at the USDA/ARS Catfish Genetics Research Unit, Stoneville, Miss.

TABLE 33

| | NWAC103 vs Norris | | | | | | 22% Protein Diet vs. 32% Protein Diet | | | | | |
|---|---|---|---|---|---|---|---|---|---|---|---|---|
| | NWAC 103 | | Norris | | | | 32% | | 22% | | | |
| | Males | Female | Males | Female | S.E. | Effects* | Males | Female | Males | Female | S.E. | Effects |
| Weight | 638 | 574 | 543 | 479 | 17.0 | Line, Sex | 638 | 574 | 613 | 540 | 23.5 | Sex |
| Gutted % | 90.4 | 89.0 | 90.8 | 89.0 | 0.15 | Sex | 90.4 | 89.1 | 89.9 | 88.5 | 0.32 | Sex |
| Headed- | 65.8 | 66.8 | 65.6 | 66.7 | 0.38 | Sex | 65.8 | 66.8 | 64.8 | 65.5 | 0.26 | Diet, |
| Shank | 36.2 | 37.8 | 35.7 | 37.2 | 0.43 | Sex | 36.2 | 37.8 | 35.7 | 36.6 | 0.26 | Diet, |
| Nugget % | 8.7 | 8.4 | 8.8 | 8.5 | 0.11 | Sex | 8.8 | 8.4 | 8.6 | 8.2 | 0.11 | Sex |

*Indicates effect is significant at P < 0.10.

The inventors have determined that optimal performance of the substantially purebred non-transgenically developed catfish of the present invention can best be attained by following recommendations as outlined below. Based on research studies evaluating the performance of NWAC103 line catfish and other research production studies, recommendations are described for: 1) broodfish care, spawning, and hatchery management, 2) fingerling culture, and 3) foodfish culture. The following recommendations are not all inclusive, but do cover critical areas for traits believed to be desirable for a commercially produced fish. In addition to the guidelines below, producers should utilize the detailed information contained in references such as “Channel Catfish Farming Handbook” by C. S. Tucker and E. H. Robinson, “Channel Catfish Fingerling Production”, “Pond Preparation for Spawning Channel Catfish”, and “Fry Pond Preparation for Rearing Channel Catfish” published by the MSU Cooperative Extension Service, each of which is fully incorporated herein by reference.

Broodfish Care Spawning and Hatchery Management

Proper management and care of broodfish is critical for high spawning success. Many factors such as water quality, stocking density, and off-season management can affect catfish reproduction. Industry averages for spawning success are estimated to be around 30-40% and egg hatching around 60%. The following guidelines will improve the probability of spawning success and fry production.

Spawning success can be as high as 20-30% in 2-year old fish and best reproduction will be realized from 3 and 4-year old fish. Broodfish larger than 10 pounds are somewhat difficult to handle and result in lower fry production per pound of broodfish.

Broodfish should be inventoried and sexed yearly during late winter while water temperatures are cool. Either a sex ratio of 1:1 or 2:1 females to males is desirable. The ratio of males to females should be closely monitored yearly because males have higher mortality than females.

Broodfish should be stocked at no more than 1,200 lbs/acre into ponds that have been drained, allowed to dry and recently re-flooded. After the spawning season, broodfish can be moved and restocked into ponds at 3,000 to 4,000 lbs/acre.

Leaving broodfish in the same pond two years in a row without draining/drying the pond or inventorying the fish often results in poor spawning success the second year and broodfish survival is unknown.

Feed a nutritionally complete floating diet with at least 28% protein at 2% of body weight/day when temperatures are above 70° F. and 1%/day with a slow-sinking pellet between 55° and 70° F. Generally no feed is offered below 50° F. Forage fish (fathead minnows and tilapia) can be added for supplemental feed and may increase spawning success.

Spawning activity will begin in the spring when water temperatures are consistently around 75° F. Maintaining optimum water quality in spawning ponds is important because low dissolved oxygen levels and excessive algae and aquatic weed growth will inhibit spawning success.

Use 50-75 spawning cans/500 females. Spawning cans can be checked every 2 days during the spawning season. Eggs should not be crowded into transport containers and do not allow transport water to become warmer than 85° F. before transport to the hatchery.

Aquatic weeds in broodfish ponds can be controlled with grass carp stocked at 25 fish/acre. Heavy aquatic vegetation may cause pH values to exceed 9.5, even in well-buffered pond water, and discourage spawning activity or cause poor egg quality.

Water for hatching eggs should be well-water with temperatures between 75° F. and 82° F. with 80° F. being optimum. Dissolved oxygen levels should be maintained above 6.0 ppm, total water hardness and alkalinity >20 ppm, pH between 7.5 and 8.5, and total gas pressure 100% of saturation or less.

Control bacterial and fungal infections on eggs by maintaining optimum water temperatures, cleaning hatchery equipment, and using formalin and iodine as needed.

If poor hatching success and unacceptable normal fry mortality occurs, send samples to diagnostic laboratories for diagnosis and treatment recommendations.

Fry will “swim-up” and begin feeding 3-4 days after hatching. Feed fry a suitable ration at least 12-24 times/day.

Fingerling Culture

Successful fingerling production requires technical skill and intensive management. Growth and survival of catfish fry to fingerling size depends on maintaining water quality, controlling disease, and providing enough feed to achieve the desired harvest-size. Survival and yield during the first growing season from fry to fingerling can be highly variable. Although, the industry average for survival of fry to fingerling has been estimated at 65% with a yield of about 3,000 lbs/acre, acute problems with disease and water quality can drastically affect survival and yield in fingerling ponds. Following recommended management protocols will improve production.

Preparation of ponds before stocking fry is critical for good survival. Fry/fingerling ponds should be drained and dried to kill all trash fish and vegetation before filling with well-water.

Fertilize ponds, check for zooplankton populations, and control predaceous insects following recommended management guidelines.

Count fry volumetrically or by weight prior to stocking into ponds. Fry can be stocked at 7-10 days old after they are actively feeding. Fry are normally stocked at 75,000 to 125,000/acre.

Stock fry into ponds with stable morning dissolved oxygen readings above 5 ppm and during the morning before water temperatures exceed 85° F. Transport fry to ponds in oxygenated tanks and acclimate fry to pond temperatures if necessary.

Vaccination of fry prior to stocking may improve survival and resistance to bacterial infections.

After stocking, fry ponds should be fed finely ground feed (usually 40-50% protein) 2-3 times daily (20-30 lbs/acre/day) until fish are observed feeding and swimming on the pond surface. Feed should be distributed around the entire perimeter of the pond.

Fry should be observed feeding within 3-5 weeks after stocking. Begin feeding a small pellet floating feed to satiation daily once the fish are actively feeding.

As fingerlings grow and feeding rates increase, water quality problems can occur. Oxygen consumption of small fish is greater than large fish, so supplemental aeration is necessary for fingerlings ponds. Addition of salt to maintain chloride levels of 100 ppm is recommended.

If fingerling mortalities are seen in ponds, send samples to diagnostic laboratories for diagnosis and recommended treatments.

At the onset of cool weather in the fall when morning pond water temperatures begin to drop below 80° F., feed fish a restricted feeding regime on alternate days or every second day. Use feed containing antibiotics (Romet7 or Terramycin7) if fish are diagnosed with bacterial infections and treatment with medicated feed is recommended by a diagnostic laboratory.

Foodfish Culture

Fingerlings are typically stocked into growout ponds at 5,000-8,000 fish/acre, and even up to 10,000 is not uncommon. Industry average mortality is estimated to be 2%/month. Fingerlings typically reach marketable size in 150-200 days and grow best above 70° F. No well-defined production schedule is used on commercial farms because food-sized fish are harvested and fingerlings stocked year-round, and ponds contain fish of various sizes.

Fingerlings handle best and should be stocked when pond water temperatures are below 70° F.

Salt should be added to ponds to maintain chloride levels >100 ppm to prevent nitrite toxicosis and enhance osmoregulation.

If fingerling mortalities are seen in ponds, the cause should be determined immediately. Send fish and water samples to diagnostic laboratories for diagnosis and recommended treatments.

### Conclusion

NWAC103 line catfish were developed and evaluated at USDA/ARS Catfish Genetics Research Unit in cooperation with the Mississippi Agricultural and Forestry Experiment Station, Thad Cochran National Warmwater Aquaculture Center, Stoneville, Miss. and jointly released to commercial producers. Results of experimental trials have shown NWAC103 catfish have excellent growth compared to other catfish currently being used by producers and are recommended for foodfish production. The growth advantage of NWAC103 catfish appears to be due to aggressive feeding behavior and higher feed consumption. Optimum growth and production of NWAC103 catfish necessitates maintaining optimum environmental conditions. Although catfish farmers utilize a variety of management practices that are specific to individual farms, there are general management recommendations developed through research that have been demonstrated to improve production efficiency. Catfish mortalities occur for a diversity of reasons at any time of the year. Cause of mortality should always be determined and remedial action taken.

The invention of this application is described above both generically, and with regard to specific embodiments. A wide variety of alternatives known to those of ordinary skill in the art can be selected within the generic disclosure, and examples are not be interpreted as limiting, unless specially so indicated. The invention is not otherwise limited, except for the recitation of the claims set forth below. All references cited herein are incorporated in their entirety.

What is claimed is:

1. A substantially purebred non-transgenically developed fish useful for breeding stock having at least one desired trait, the breeding stock fish being produced by a process comprising:
selecting a subgroup of potential breeder fish from a population of same-species fish;
identifying breeder fish from within said subgroup of potential breeder fish whereby said identifying is accomplished through genetic identification of tissue samples taken from said breeder fish and compared to at least a partial DNA fingerprint of fish known to have said at least one desired trait;
breeding said identified breeder fish to produce a substantially purebred non-transgenically developed fish having the at least one desired trait.

2. The fish of claim 1, wherein the tissue samples are blood.

3. The fish of claim 1, wherein the fish is a channel catfish.

4. The fish of claim 1, wherein the fish produced is a NWAC 103 catfish.

5. The fish of claim 1, wherein substantially purebred is at least 90% purebred based on said process.

6. The fish of claim 1, wherein substantially purebred is at least 95% purebred based on said process.

7. The fish of claim 1, wherein substantially purebred is at least 97% purebred based on said process.

8. The fish of claim 1, wherein said genetic identification of tissue samples is accomplished by comparison of microsatellite loci of said tissue sample with said DNA fingerprint.

9. The fish of claim 8, wherein said microsatellite loci include locus selected from the group consisting of IpCG0002, IpCG0032, IpCG0035, IpCG0038, IpCG0070, IpCG0128, IpCG0189, IpCG0195, IpCG0211, IpCG0256, IpCG0273, or combinations thereof, wherein IpCG0002 is identified by primers SEQ ID NO.1 and SEQ ID NO. 2, IpCG0032 is identified by primers SEQ ID NO.3 and SEQ ID NO. 4, IpCG0035 is identified by primers SEQ ID NO.5 and SEQ ID NO. 6, IpCG0038 is identified by primers SEQ ID NO.7 and SEQ ID NO. 8, IpCG0070 is identified by primers SEQ ID NO.9 and SEQ ID NO. 10, IpCG0128 is identified by primers SEQ ID NO.11 and SEQ ID NO.12, IpCG0189 is identified by primers SEQ ID NO.13 and SEQ ID NO. 14, IpCG0195 is identified by primers SEQ ID NO.15 and SEQ ID NO. 16, IpCG0211 is identified by primers SEQ ID NO.17 and SEQ ID NO. 18, IpCG0256 is identified by primers SEQ ID NO.19 and SEQ ID NO. 20, IpCG0273 is identified by primers SEQ ID NO.21 and SEQ ID NO. 22.

10. A substantially purebred non-transgenically developed fish having at least one desired trait and useful as breeding stock, the fish being produced by a process comprising:
selecting a first subgroup of fish, which demonstrate at least one desired trait, from a population of same-species fish;
taking a first set of discrete tissue samples from a plurality of fish within said subgroup;
isolating genomic DNA from said first samples;
amplifying said genomic DNA of said first samples using polymerase chain reaction;
determining DNA fragment size of said first samples by electrophoresis;
characterizing variation in microsatellite loci for said first samples for at least one generation of fish demonstrating said at least one desired trait;
selecting a plurality of microsatellite loci of said first samples, all of which are common to only those fish demonstrating said at least one desired trait;
selecting a potential breeder fish subgroup from said population of same-species fish;
taking a second set of discrete tissue samples from a plurality of fish within said potential breeder fish subgroup;
isolating genomic DNA from said discrete tissue samples of the potential breeder subgroup;
amplifying said genomic DNA of the potential breeder tissue samples using polymerase chain reaction;
determining DNA fragment size for said breeder tissue samples by electrophoresis to create at least a partial DNA fingerprint for each of said breeder tissue samples;
determining which of said breeder tissue samples contain all of said common microsatellite loci and identifying the fish from which those breeder tissue samples having all common microsatellite loci were taken as breeder fish;
breeding said identified breeder fish to produce a substantially purebred non-transgenically developed breeding stock fish having the at least one desired trait.

11. The fish of claim 10, wherein the tissue samples are blood.

12. The fish of claim 10, wherein the fish is a channel catfish.

13. The fish of claim 10, wherein the fish produced is a NWAC 103 catfish.

14. The method of claim 10, wherein said microsatellite loci include locus selected from the group consisting of IpCG0002, IpCG0032, IpCG0035, IpCG0038, IpCG0070, IpCG0128, IpCG0189, IpCG0195, IpCG0211, IpCG0256, IpCG0273, or combinations thereof, wherein IpCG0002 is identified by primers SEQ ID NO.1 and SEQ ID NO. 2, IpCG0032 is identified by primers SEQ ID NO.3 and SEQ ID NO. 4, IpCG0035 is identified by primers SEQ ID NO.5 and SEQ ID NO. 6, IpCG0038 is identified by primers SEQ ID NO.7 and SEQ ID NO. 8, IpCG0070 is identified by primers SEQ ID NO.9 and SEQ ID NO. 10, IpCG0128 is identified by primers SEQ ID NO.11 and SEQ ID NO.12, IpCG0189 is identified by primers SEQ ID NO.13 and SEQ ID NO. 14, IpCG0195 is identified by primers SEQ ID NO.15 and SEQ ID NO. 16, IpCG0211 is identified by primers SEQ ID NO.17 and SEQ ID NO. 18, IpCG0256 is identified by primers SEQ ID NO.19 and SEQ ID NO. 20, IpCG0273 is identified by primers SEQ ID NO.21 and SEQ ID NO. 22.

15. A method of selecting breeding stock for the production of a substantially purebred non-transgenically developed fish having at least one desired trait and useful as breeding stock, said method comprising:
selecting potential breeding stock having said at least one desired trait from a population of same-species fish;
taking a tissue sample from said potential breeding stock and comparing the genotype of said potential breeding stock to at least a partial DNA fingerprint of fish known to have said at least one desired trait;
identifying breeding stock as those fish, which provided tissue samples that corresponded to specific microsatellite loci known to be found in said DNA fingerprint.

16. The method of claim 15, wherein the tissue samples are blood.

17. The method of claim 15, wherein the fish is a channel catfish.

18. The method of claim 15, wherein the fish produced is a NWAC 103 catfish.

19. The method of claim 15, wherein said microsatellite loci include locus selected from the group consisting of IpCG0002, IpCG0032, IpCG0035, IpCG0038, IpCG0070, IpCG0128, IpCG0189, IpCG0195, IpCG0211, IpCG0256, IpCG0273, or combinations thereof, wherein IpCG0002 is identified by primers SEQ ID NO.1 and SEQ ID NO. 2, IpCG0032 is identified by primers SEQ ID NO.3 and SEQ ID NO. 4, IpCG0035 is identified by primers SEQ ID NO.5 and SEQ ID NO. 6, IpCG0038 is identified by primers SEQ ID NO.7 and SEQ ID NO. 8, IpCG0070 is identified by primers SEQ ID NO.9 and SEQ ID NO. 10, IpCG0128 is identified by primers SEQ ID NO.11 and SEQ ID NO.12, IpCG0189 is identified by primers SEQ ID NO.13 and SEQ ID NO. 14, IpCG0195 is identified by primers SEQ ID NO.15 and SEQ ID NO. 16, IpCG0211 is identified by primers SEQ ID NO.17 and SEQ ID NO. 18, IpCG0256 is identified by primers SEQ ID NO.19 and SEQ ID NO. 20, IpCG0273 is identified by primers SEQ ID NO.21 and SEQ ID NO. 22.
